SEQUENCE LISTING (4 sequences)

SEQ ID NO: 1 - protein, 115 aa, synthetic construct
LRAFEDEKKARMGVVECAKHELLQPFNVLYEKEGEFVAQFKFTVLLMPNGPMRITSGPFEPDLYKSEMEVQDAELKALLQSSASRKTQKKKKKKASKTAENATSGETLEENEAGD

SEQ ID NO: 2 - protein, 394 aa, synthetic construct
MSGEDEQQEQTIAEDLVVTKYKMGGDIANRVLRSLVEASSSGVSVLSLCEKGDAMIMEETGKIFKKEKEMKKGIAFPTSISVNNCVCHFSPLKSDQDYILKEGDLVKIDLGVHVDGFIANVAHTFVVDVAQGTQVTGRKADVIKAAHLCAEAALRLVKPGNQNTQVTEAWNKVAHSFNCTPIEGMLSHQLKQHVIDGEKTIIQNPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKKASKTAENATSGETLEENEAGD

SEQ ID NO: 3 - DNA, 348 bp, synthetic construct
  source 1..348: mol_type = other DNA
ttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

SEQ ID NO: 4 - DNA, 1185 bp, synthetic construct
  source 1..1185: mol_type = other DNA
atgtcgggcgaggacgagcaacaggagcaaactatcgctgaggacctggtcgtgaccaagtataagatggggggcgacatcgccaacagggtacttcggtccttggtggaagcatctagctcaggtgtgtcggtactgagcctgtgtgagaaaggtgatgccatgattatggaagaaacagggaaaatcttcaagaaagaaaaggaaatgaagaaaggtattgcttttcccaccagcatttcggtaaataactgtgtatgtcacttctcccctttgaagagcgaccaggattatattctcaaggaaggtgacttggtaaaaattgaccttggggtccatgtggatggcttcatcgctaatgtagctcacacttttgtggttgatgtagctcaggggacccaagtaacagggaggaaagcagatgttattaaggcagctcacctttgtgctgaagctgccctacgcctggtcaaacctggaaatcagaacacacaagtgacagaagcctggaacaaagttgcccactcatttaactgcacgccaatagaaggtatgctgtcacaccagttgaagcagcatgtcatcgatggagaaaaaaccattatccagaatcccacagaccagcagaagaaggaccatgaaaaagctgaatttgaggtacatgaagtatatgctgtggatgttctcgtcagctcaggagagggcaaggccaaggatgcaggacagagaaccactatttacaaacgagacccctctaaacagtatggactgaaaatgaaaacttcacgtgccttcttcagtgaggtggaaaggcgttttgatgccatgccgtttactttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

(19) United States
(12) Patent Application Publication
(10) Pub. No.: US 2024/0325490 A1
(43) Pub. Date: Oct. 3, 2024

(54) PHARMACEUTICAL COMPOSITION FOR PREVENTING OR TREATING ISCHEMIC CEREBROVASCULAR DISEASE

(71) Applicant: Research & Business Foundation Sungkyunkwan University, Suwon-si (KR)

(72) Inventors: Jee Yin AHN, Seoul (KR); lnwoo HWANG, Suwon-si (KR); Byeong Seong KIM, Suwon-si (KR)

(73) Assignee: Research & Business Foundation Sungkyunkwan University, Suwon-si (KR)

(21) Appl. No.: 18/437,974

(22) Filed: Feb. 9, 2024

(30) Foreign Application Priority Data
Feb. 10, 2023 (KR) 10-2023-0018316

(51) Int. Cl. A61K 38/17; A61K 48/00; A61P 9/10; A61P 25/28

(52) U.S. Cl. CPC A61K 38/1709; A61K 48/0033; A61P 9/10; A61P 25/28

(57) ABSTRACT

Disclosed is a pharmaceutical composition, containing ErbB3-binding protein 1 (EBP1) protein or a polynucleotide encoding Ebp1 protein as an active ingredient, for preventing and treating ischemic cerebrovascular disease, wherein the pharmaceutical composition has excellent effect of suppressing neuronal damage caused by ischemia by promoting mitophagy and thus can be advantageously used as an agent for preventing or treating ischemic cerebrovascular disease.

### CROSS-REFERENCE TO RELATED APPLICATION

This application claims the benefit under 35 USC 119(a) of Korean Patent Application No. 10-2023-0018316, filed with the Korean Intellectual Property Office on Feb. 10, 2023, the disclosure of which is incorporated herein by reference in its entirety.

### INCORPORATION-BY-REFERENCE OF MATERIAL SUBMITTED BY U.S.P.T.O. EFS-WEB

The instant application contains a Sequence Listing which is being submitted in computer readable form via the United States Patent and Trademark Office eFS-WEB system and which is hereby incorporated by reference in its entirety for all purposes. The XML file submitted herewith contains a 6.39 KB file (NewApp_0421930011_SeqListingAsFiled).

### BACKGROUND OF THE INVENTION

### 1. Field of the Invention

The present disclosure was made with the support of the Ministry of Health and Welfare, Republic of Korea, under Project No. HU21C0157000022, Project Identification No. 1465035886, which was conducted in the research project named “Identification of neural degenerative mechanism in Alzheimer's disease-like pathological phenotype (loss of function Ebp1 mouse) model and Discovery of new treatment targets for AD” in the research program titled “Dementia Overcoming Research and Development Program”, by Sungkyunkwan University, under the management of the Korea Health Industry Development Institute, from 1 Apr. 2021 to 31 Dec. 2023.

The present disclosure was also made with the support of the Ministry of Science and ICT of the Republic of Korea, under Project No. 2016R1A5A2945889, Project Identification No. 1711164024, which was conducted in the research project named “Single Cell Network Research Center” in the research program titled “Collective Research Support”, by School of Medicine, Sungkyunkwan University, under the management of the National Research Foundation of Korea, from 1 Dec. 2016 to 31 Aug. 2023.

This application claims priority and the benefit of Korean Patent Application No. 10-2023-0018316 filed in the Korean Intellectual Property Office on 10 Feb. 2023, the disclosure of which is incorporated herein by reference.

The present disclosure relates to a pharmaceutical composition for preventing or treating ischemic cerebrovascular disease. More specifically, the present disclosure relates to a pharmaceutical composition for preventing or treating ischemic cerebrovascular disease, the pharmaceutical composition containing, as an active ingredient, ErbB3-binding protein 1 (Ebp1) protein capable of suppressing brain damage in the early stage of ischemic cerebrovascular disease or a polynucleotide encoding Ebp1 protein.

### 2. Description of the Prior Art

Stroke is a disease that causes brain damage due to the interruption of blood flow to the brain. During the first 7 days (acute phase) of the onset of stroke, the death of damaged brain cells occurs. During the period of 3 months up to 6 months (sub-acute phase), rehabilitation is carried out to restore the functions of the brain damaged in the early stage of onset. The degree of recovery varies from individual to individual, but the recovery to the same as before is impossible. Existing therapeutic agents have been studied according to the mechanisms that enable treatment according to the sub-acute phase, but more effective treatment can be made if the brain damage occurring during the acute phase can be fundamentally prevented.

### PRIOR ART DOCUMENT

### Non-Patent Document

Hwang et al., Cerebellar dysfunction and schizophrenia-like behavior in Ebp1-deficient mice., Molecular Psychiatry 27, 2030-2041 (2022).

### SUMMARY OF THE INVENTION

The present inventors made intensive research efforts to develop a pharmaceutical composition for preventing or treating ischemic cerebrovascular disease, the composition being capable of suppressing brain damage in the early stage of the onset of ischemic cerebrovascular disease, such as stroke. As a result, the present inventors established that the increase in the expression of Ebp1 gene or protein in stroke model animals can suppress brain damage caused by stroke, and thus completed present disclosure.

Accordingly, an aspect of the present disclosure is to provide a pharmaceutical composition, containing ErbB3-binding protein 1 (EBP1) protein or a polynucleotide encoding EBP1 protein as an active ingredient, for preventing or treating ischemic cerebrovascular disease.

Another aspect of the present disclosure is to provide a method for treating ischemic cerebrovascular disease in a non-human animal, the method including administering ErbB3-binding protein 1 (EBP1) protein or a polynucleotide encoding EBP1 protein to a subject.

In accordance with an aspect of the present disclosure, there is provided a pharmaceutical composition, containing ErbB3-binding protein 1 (EBP1) protein or a polynucleotide encoding EBP1 protein as an active ingredient, for preventing or treating ischemic cerebrovascular disease.

In accordance with another aspect of the present disclosure, there is provided a method for treating ischemic cerebrovascular disease, the method including administering to a subject ErbB3-binding protein 1 (EBP1) protein or a polynucleotide encoding EBP1 protein.

Since the method for preventing or treating ischemic cerebrovascular disease uses ErbB3-binding protein 1 (EBP1) protein or a polynucleotide encoding EBP1 protein, which is an active ingredient of the pharmaceutical composition according to an aspect of the present disclosure, in common with the pharmaceutical composition, the overlapping contents therebetween are applied to the method of the present disclosure in the same manner, and the overlapping description therebetween is omitted in order to avoid excessive complexity of the present specification.

As used herein, the term “ErbB3-binding protein 1 (Ebp1)” refers to a gene that is located in chromosome band 12q13.2, over-expressed in various cancer cells, such as brain tumors and breast cancer, and is strongly expressed during brain development or in adults.

In an embodiment of the present disclosure, the EBP1 protein includes the amino acid sequence represented by of SEQ ID NO: 1.

In an embodiment of the present disclosure, the EBP1 protein includes the amino acid sequence represented by of SEQ ID NO: 2.

In an embodiment of the present disclosure, the polynucleotide encoding EBP1 protein includes the amino acid sequence represented by of SEQ ID NO: 3.

In an embodiment of the present disclosure, the polynucleotide encoding EBP1 protein includes the nucleotide sequence represented by of SEQ ID NO: 4.

The present inventors found that Ebp1 expression increased in stroke patients. In addition, protein signals inducing apoptosis increase in mitochondria damaged by reactive oxygen species increased due to stroke, and it was experimentally verified that such a situation was further increased in brain-specific Ebp1 knockout mice. Therefore, the present inventors hypothesized that the expression of Ebp1 gene can suppress brain damage caused by stroke, and validated the hypothesis through experiments.

In an embodiment of the present disclosure, the Ebp1 gene shows a rapidly increasing expression in the damaged mitochondrial outer membrane, and the ubiquitination to Ebp1 is increased by Parkin, E3 ligase. This increases mitophagy, inducing a rapid removal of apoptosis factors before release from damaged mitochondria.

As used herein, the term “mitophagy” refers to an intracellular degradation mechanism that removes damaged or unnecessary mitochondria, wherein in the event of mitochondrial damage, autophagosomes are formed with surrounding of membranes and then fused with lysosomes to selective remove damaged mitochondria. It is known that such mitophagy is important in regulating mitochondrial functions and maintaining tissue functions in several cells including neurons. As the abnormality in mitophagy has been reported for a wide range of human diseases, for example, peripheral neuropathy, heart disease, metabolic disease, and cancer, including degenerative brain diseases such as Parkinson's disease, researchers are increasingly interested in the role of mitophagy in human diseases and its availability in the treatment of diseases.

Mitophagy is known to have a neuroprotective function even in ischemic cerebral diseases. However, it is not revealed how mitophagy is activated in ischemic brains to alleviate neuronal death.

In an embodiment of the present disclosure, the present inventors verified that ischemia reperfusion (IR)-injury induced a transient increase in EBP1 expression through K376 ubiquitination by PARKIN, but CA1 neurons were selectively susceptible to IR injury due to the depletion of EBP1 or the damage to K376 ubiquitination in Ebp1.

In a specific embodiment of the present disclosure, the increase in EBP1 and K376 ubiquitination preceded or coincided with an increase in the expression of the autophagy marker LC3II and a decrease in the expression of TOMM 20 and cargo adapter P62 contributing to mitophagy induction.

Furthermore, the present inventors validated that PARKIN-mediated EBP1 K376 ubiquitination was involved in K63-linked chains and appeared to regulate mitophagy induction by recruiting P62, subsequently preventing neuronal death in the ischemic injury.

EBP1 in the present disclosure is known to be associated as an anti-apoptosis protein in neurons or a specific type of cancer, such as glioblastoma, but cerebral ischemic injury has not been studied or recognized during mitophagy.

The present inventors validated that the upregulation of EBP1 in the early frame of ischemic damage prevented neuronal death, decreased the brain infarct volume, and alleviated motor and cognitive impairments. Notably, EBP1 effectively induced mitophagy in the ischemic brain, whereas the brain-specific deletion of EBP1 in mice did not lead to mitophagy induction after MCAO and in CCCP-treated cells. Accordingly, EBP1 of the present disclosure is considered to be translocated to the mitochondria upon mitochondrial damage and contribute to mitophagy enhancement. This prevents neuronal death in the early period of damage, but the sustained IR injury no longer preserves the expression of EBP1, indicating that hippocampal neurons cannot survive.

In cerebral IR injury, an attention has been conventionally focused on the PINK-PARKIN pathway, which is known to be a sensor of mitochondria damage. For example, research has been conducted on hypoxic postconditioning-mediated neuroprotection against cerebral ischemia occurring through mitochondria ubiquitination and mitophagy activation triggered by activated PINK-PARKIN. The mitochondria translocation of PARKIN upon mitochondria damage mediates mitochondrial priming and is a crucial step for preparing the mitochondria for autophagy recognition to promote autophagic removal. Interestingly, PARKIN-mediated polyubiquitination on mitochondria leaded to OMM protein degradation through proteasome, and the removal of OMM protein by the PARKIN-independent proteasome system is important for mitophagy.

In addition, PARKIN-mediated ubiquitination contributes to the recruitment of ubiquitin-binding autophagy receptors, such as P62/SQSTM1, which is known to connect the ubiquitin system to the autophagic machinery. These receptors isolate particular ubiquitination components by autophagosomes before transfer to lysosomes for degradation, thereby ensuring mitophagy. The present inventors verified corresponding to these results that EBP1 is a novel substrate for PARKIN in the mitochondria damaged after MCAO.

In a more specific embodiment of the present disclosure, PARKIN was connected to K63-linked ubiquitination of EBP1 K376, and this ubiquitination allowed EBP1 accumulation at the damaged mitochondria and acted as a linker for the adaptor protein P62, leading to efficient mitophagy induction. However, the in vivo delivery of K376A mutant, which has a defect in ubiquitination, did not lead to mitophagy induction and did not prevent the neuronal death and behavioral defects under MCAO, indicating that the absence of PARKIN-mediated K63-linked-ubiquitination on EBP1 hindered the recruitment of the adaptor protein P62.

Mitochondria dysfunction is involved in multiple pathophysiological processes after cerebral ischemia. The mitophagy in the present disclosure, which is an endogenous adaptive response, is known to affect a neuronal fate in the ischemic brain through selective mitochondria turnover.

The present inventors proposed a mechanism whereby the increase in EBP1 ubiquitination at K376 by PARKIN upon mitochondrial damage promoted mitophagy and attenuated neuronal death in clinically relevant ischemia models. The induction of ischemia triggered a transient increase in EBP1 expression and its translocation to the mitochondria, where EBP1 K376 ubiquitination by PARKIN, resulting in the recruitment of the adaptor protein P62, thereby promoting mitophagy in neurons before neuronal death. Therefore, the mitophagy induction promotes neuroprotection in response to ischemic stimulation.

The present inventors verified a neuroprotective molecular target of PARKIN-mediated mitophagy connected to cerebral IR injury, through PARKIN-mediated EBP1 ubiquitination. The present inventors further showed an inverse causal relationship between the impairment of PARKIN-dependent EBP1 ubiquitination and mitophagy induction as well as neuronal death. Therefore, it can be derived from the results that the targeted control of mitophagy during the appropriate time frame is a therapeutic intervention for cerebral ischemia.

In an embodiment of the present disclosure, the polynucleotide encoding Ebp1 protein induces over-expression of Ebp1 in host cells with which host cells were transfected. The host cell is, for example, a neuron, and the origin of the neuron may be the central nervous system, specifically the brain, more specifically the cerebellum, mid-brain, cerebellum, and hepatic brain, but is not limited thereto.

In an embodiment of the present disclosure, the polynucleotide encoding Ebp1 protein induces transient or persistent over-expression of Ebp1 in transformed host cells.

As used herein, the term “over-expression” refers to an increased expression of a gene of interest. The over-expression has a meaning encompassing increasing transcription and/or translation of a gene of interest through a method where the gene of interest is cloned into an expression vector and transfected into cells, and the other methods.

In an embodiment of the present disclosure, the polynucleotide is naked DNA, or is contained in a gene carrier.

As used herein, the term “naked DNA” refers to DNA that is not associated with proteins, lipids, or DNA to help protect itself.

As used herein, the term “gene carrier” refers to a DNA molecule that functions to deliver an exogenous DNA fragment, which is inserted into its own DNA.

In an embodiment of the present disclosure, the gene carrier is a vector.

As used herein, the term “vector” refers to any means for expressing a target gene in a host cell, and examples thereof include phagemid vectors, plasmid vectors, cosmid vectors, viral vectors, such as a bacteriophage vector, an adenoviral vector, a retroviral vector, a lentiviral vector, and an adeno-associated viral vector, but are not limited thereto.

In an embodiment of the present disclosure, an adeno-associated virus (AAV) for the adeno-associated viral vector is AAV1, AAV2, AAV5, or AAV6, but is not limited thereto.

In an embodiment of the present disclosure, the adeno-associated virus (AAV) is AAV2.

In an embodiment of the present disclosure, the vector may be a recombinant vector.

In an embodiment of the present disclosure, the plasmid may be a recombinant plasmid.

In an embodiment of the present disclosure, the polynucleotide encoding Ebp1 protein in the vector of the present disclosure is operatively linked to a promoter.

As used herein, the expression “operatively linked” refers to functional linking between a nucleic acid expression control sequence (e.g., a promoter, a signal sequence, or an array of transcription factor binding sites) and another nucleic acid sequence, whereby the control sequence directs the transcription and/or translation of the another nucleic acid sequence.

The vector or recombinant vector of the present disclosure may be constructed by various methods known in the art, and specific methods therefor are disclosed in Sambrook et al., Molecular Cloning, A Laboratory Manual, Cold Spring Harbor Laboratory Press (2001), which is incorporated herein by reference.

The vector of the present disclosure may be typically constructed as a vector for cloning or a vector for expression. The vector for cloning of the present disclosure may be constructed by using a prokaryotic or eukaryotic cell as a host. Additionally, the vector for cloning of the present disclosure includes a multiple cloning site (MCS).

As used herein, the term “promoter” promotes the expression of a gene to be transfected, and the promoter may further include not only a basal element necessary for transcription, but also an enhancer that may be used to promote and regulate the expression.

For example, in cases where the vector of the present disclosure is an expression vector and an eukaryotic cell is used as a host cell, promoters derived from the genomes of mammalian cells (e.g., a gene to be transfected, and the promoter may further include not only a basic element necessary for transcription, but also an enhancer that may be used to promote and regulate the expression.) or promoters derived from mammalian viruses (e.g., adenovirus late promoter, vaccinia virus 7.5K promoter, SV40 promoter, cytomegalovirus promoter, HSV tk promoter, mouse mammary tumor virus (MMTV) promoter, HIV LTR promoter, Moloney virus promoter, Epstein-Barr virus (EBV) promoter, and Rous sarcoma virus (RSV) promoter) may be used, and these typically have a polyadenylated sequence as a transcription termination sequence.

In an embodiment of the present disclosure, the promoter may be an elongation factor 1 alpha (EF1α) promoter or a cytomegalovirus (CMV) promoter, but is not limited thereto.

The vector of the present disclosure may be fused with the other sequences to facilitate the purification of proteins expressed therefrom. Examples of the sequences that are fused include glutathione S-transferase (Pharmacia, USA), maltose binding proteins (NEB, USA), FLAG (IBI, USA), 6×His (hexahistidine; Quiagen, USA), and the like.

The vector of the present disclosure includes, as a selective marker, an antibiotic agent-resistant gene that is commonly used in the art, and examples thereof include resistant genes against ampicillin, gentamicin, carbenicillin, chloramphenicol, streptomycin, kanamycin, geneticin, neomycin, and tetracycline.

Optionally, the vector may additionally deliver a gene encoding a reporter molecule (e.g., luciferase and glucuronidase).

In an embodiment of the present disclosure, the ischemic cerebrovascular disease is selected from the group consisting of transient ischemic attack, reversible ischemic neurological deficit, and obstructive stroke, but is not limited thereto.

In an embodiment of the present disclosure, the obstructive stroke is caused by blood clots or embolisms.

In an embodiment of the present disclosure, the stroke is an acute stroke within 7 days of the onset.

As used herein, the term “prevention” refers to any action that inhibits or delays the onset of the brain disease in a subject by the administration of the composition according to the present disclosure, and the term “treatment” refers to any action that alleviates the brain disease or advantageously change the disease status in a subject by the administration of the composition.

In an embodiment of the present disclosure, the subject is a mammal. Examples of the mammal include cows, horses, sheep, pigs, goats, camels, antelopes, dogs, and cats, as well as primates including humans. However, as described later, humans are excluded for the prevention or treatment method in accordance with another aspect of the present disclosure.

The pharmaceutical composition of the present disclosure may further contain an appropriate carrier, excipient, or diluent according to a commonly used method.

Examples of the carrier, excipient, and diluent that may be contained in the composition of the present disclosure may include lactose, dextrose, sucrose, sorbitol, mannitol, xylitol, erythritol, maltitol, starch, acacia gum, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and a mineral oil.

The composition of the present disclosure may be formulated in an oral dosage form, such as a powder, granules, a tablet, a capsule, a suspension, an emulsion, a syrup, or an aerosol, or in the form of an external preparation, a suppository, and a sterile injectable solution, according to a commonly used method for each form.

Specifically, the composition may be formulated by using a diluent or excipient, such as a filler, extender, binder, humectant, disintegrant, or surfactant. Solid preparations for oral administration may include a tablet, a pill, a powder, granules, a capsule, and the like, and these solid preparations may be prepared by adding, to the active ingredient, at least one excipient, for example, starch, calcium carbonate, sucrose, lactose, gelatin, or the like. Additionally, lubricants, such as magnesium stearate and talc, may be used in addition to simple excipients. Liquid preparations for oral administration correspond to a suspension, an oral solution, an emulsion, a syrup, and the like, and may contain not only frequently used simple diluents, such as water and liquid paraffin, but also several types of excipients, for example, a humectant, a sweetener, a flavor, and a preservative. Preparations for parenteral administration include a sterile aqueous solution, a non-aqueous solvent, a suspension, an emulsion, a freeze-dried preparation, and a suppository. Examples of the non-aqueous solvent and suspension may include propylene glycol, polyethylene glycol, a vegetable oil such as olive oil, an injectable ester such as ethyl oleate, and the like. Examples of a base for the suppository may include Witepsol, Macrogol, Tween 61, cocoa butter, laurin butter, glycerogelatin, and the like.

A preferable dose of the active ingredient of the present disclosure may vary depend on the condition, age, or weight of a patient, the severity of disease, form of a drug, and a route and period of administration, but may be selected as appropriate by a person skilled in the art. However, for desirable efficacy, the active ingredient of the present disclosure may be administered at 0.0001 to 100 mg/kg, and preferably, 0.001 to 100 mg/kg, in a single dose or divided into multiple doses per day. The active ingredient of the present disclosure needs to be present at an amount of 0.0001-10 wt %, and preferably 0.001-1 wt % relative to the total weight of the entire composition.

The pharmaceutical administration form of the active ingredient of the present disclosure may also be used in a form of pharmaceutically acceptable salts thereof, and may be used alone or in combination or in an appropriate set with other pharmaceutically active ingredients.

The pharmaceutical composition of the present disclosure may be administered to a subject through various routes. All modes of administration may be expected, and for example, administration may be conducted orally, abdominally, rectally, or through an intravenous, arterial, muscular, inhalation, transdermal, subcutaneous, intradermal, intrauterine, intracerebral, intrathecal, or intracerebroventricular injection.

According to the present disclosure, the present disclosure provides a pharmaceutical composition, containing ErbB3-binding protein 1 (EBP1) protein or a polynucleotide encoding EBP1 protein as an active ingredient, for preventing or treating ischemic cerebrovascular disease.

The pharmaceutical composition of the present disclosure has excellent effect of suppressing neuronal damage caused by ischemia by promoting mitophagy, and thus can be advantageously used as an agent for preventing or treating ischemic cerebrovascular disease.

### BRIEF DESCRIPTION OF THE DRAWINGS

The above and other aspects, features and advantages of the present disclosure will be more apparent from the following detailed description taken in conjunction with the accompanying drawings.

FIG. 1A confirms the increase in Ebp1 expression in the blood of stroke patients and the same increase in Ebp1 in human pericytes under the OGD situation in the GSE data set. FIG. 1B confirms the increases in expressions of Ebp1 RNA and protein in the hippocampus after stroke was induced in wild-type mice. FIG. 1C confirms the increase in Ebp1 expression in the hippocampus, by tissue staining.

FIG. 2A shows a timeline of experiment progress after inducing a stroke model to Ebp1 knockout mice. FIG. 2B confirms that brain damage was further increased due to stroke in the Ebp1 knock-out mouse. FIG. 2C confirms apoptosis in the hippocampus by tissue staining (Tunel, active-Caspase3). FIG. 2D confirms the overall infarct volume using MRI after MCAO (stroke). FIG. 2E is a diagram showing the survival rate of mice after MCAO depending on the presence or absence of Ebp1. FIG. 2F is a schematic diagram of a stroke (MCAO) occurrence test after injection of AAV expressing Ebp1 and a diagram confirming EBP1 expression in the hippocampus to confirm the neuroprotective effect of Ebp1. FIG. 2G is a diagram showing the size of cerebral infarction in MCAO induced after AAV-EBP1 injection. FIG. 2H is a diagram showing TUNEL-positive cells in MCAO induced after AAV-EBP1 injection. FIG. 2I is a diagram showing MAP2 positive cells in MCAO induced after AAV-EBP1 injection.

FIG. 3A confirms that Ebp1 increased after stroke was translocated to the mitochondria. FIG. 3B confirms the same translocation to the mitochondria as mentioned above, by cell staining of primary hippocampal neurons. FIG. 3C confirms the same results as in FIG. 3B in SH-SY5Y cells. FIG. 3D confirms that mitophagy was reduced in Ebp1 MEF null cells. FIG. 3E confirms the reduction of mitophagy with the increases in apoptosis factors in knockout mice, by immunoblotting. FIG. 3F confirms that mitophagy was reduced in knockout, while staining capable of checking mitophagy was used after hippocampal slice culture. FIG. 3G confirms that mitophagy was induced in primary hippocampal neurons lacking EBP1 using CCCP.

FIG. 4A confirms that the Ebp1 ubiquitin was increased by stroke induction. FIG. 4B confirms that the Ebp1 ubiquitin was increased at the initial stage of stroke, corresponding to an acute situation. FIG. 4C confirms that the interaction with the representative E3 ligase Parkin for mitophagy induction was increased. FIG. 4D confirms that the over-expression of Parkin increased the Ebp1 ubiquitin. FIG. 4E confirms by cell staining that all of Ebp1/Parkin/UB were present at the same site. FIG. 4F confirms by cell staining that Ebp1 was present along with mitophagy occurring in the mitochondria. FIG. 4G confirms that the absence of Ebp1 inhibited mitophagy induction although Parkin was present in Ebp1 MEF null cells.

FIG. 5A confirms that the ubiquitin of Ebp1 increased by Parkin was K63 that acts in signaling. FIG. 5B confirms that Parkin ubiquitinated Ebp1 K376. FIG. 5C confirms through in vitro ubiquitination assay that Ebp1 was a Parkin target protein. FIG. 5D confirms that Ebp1 ubiquitination was a signal important for the translocation to the mitochondria. FIG. 5E confirms that an Ebp1 K376A mutant could not increase mitophagy even when over-expressed. FIG. 5F shows the same results as in FIG. 5E by staining using mitophagy antibodies. FIG. 5G confirms co-localization of Ebp1 and P62 in CA1 after stroke modeling of wild-type mice. FIG. 5H confirms that there was a difference in the binding affinity between P62 and the mitochondria depending on the presence or absence of Ebp1 after stroke induction in wild-type and knockout mice. FIG. 5I shows that Ebp1 mutants had reduced binding affinity with P62. FIG. 5J shows that the ubiquitin of Ebp1 was an essential element for the binding with P62.

FIG. 6A confirms that the over-expression of Ebp1-WT inhibited the brain damage but the mutant did not inhibit wherein Ebp1-WT and K376A were injected into wild-type mice to induce stroke. FIG. 6B confirms apoptosis in the same experimental conditions as in FIG. 6A, by Tunel staining. FIG. 6C confirms the Ebp1/P62 interaction under the same experimental conditions as in FIG. 6A, by the PLA technique. FIG. 6D confirms the interaction of P62/mitochondria according to Ebp1 over-expression. FIG. 6E confirms the degree of mitophagy according to Ebp1 over-expression. FIG. 6F confirms the increased ubiquitin in Ebp1 WT but non-increased ubiquitin in K376A even in the mouse hippocampus as in cells. FIG. 6G confirms the increased P62 interaction in WT but non-increased P62 interaction in K376A even in mice. FIG. 6H shows that behavioral tests were performed in the stroke modeling after injection of AAV-Ebp1 WT and K376A in wild-type mice. FIG. 6I confirms the degree of recovery of motility by measuring the total travel distance in the open field test. FIG. 6J shows the measurement of learning & memory, which are unique functions of the hippocampus in the noble object recognition test. FIG. 6K confirms that pathological symptoms were alleviated in Ebp1 WT but not in the mutant as in the behavioral experiments, wherein the functions of the hippocampus were measured in the Y-maze test. FIG. 6L confirms 280-394 amino acids, corresponding to a small unit capable of binding to P62 to induce mitophagy in the Ebp1 protein.

### DETAILED DESCRIPTION OF THE EXEMPLARY EMBODIMENTS

Hereinafter, the present disclosure will be described in more detail with reference to exemplary embodiments. These exemplary embodiments are provided only for the purpose of illustrating the present disclosure in more detail, and therefore, according to the purpose of the present disclosure, it would be apparent to a person skilled in the art that these exemplary embodiments are not construed to limit the scope of the present disclosure.

### EXAMPLES

### Materials and Methods

### Animals

Mouse Ebp1 gene is located on chromosome 10 (NM_011119). The Ebp1-knockout mice were generated in collaboration with genOway (Lyon, France). To achieve neuron-specific expression of Ebp1, homozygous mutants of Ebp1 alleles (Ebp1^{flox/flox}) were crossed with CamKII-Cre mice. All animal experiments were reviewed and approved by the Institutional Animal Care and Use Committee (IACUC) of Sungkyunkwan University School of Medicine (SUSM, SKKUIACUC 2022-02-27-1). All experimental procedures were carried out in accordance with the regulations of the IACUC guidelines at Sungkyunkwan University.

### Antibodies

Anti-EBP1 (ab186846) was acquired from Abcam. Anti-EBP1 (ABE43) was acquired from Sigma-Aldrich. Anti-NeuN (ABN78) was acquired from Millipore. Anti-P62 (sc-28359), UB (sc-8017), ACTIN (sc-8432), GST (sc-138), GFP (sc-9996), and PARKIN (sc-32282) were acquired from Santa Cruz Biotechnology. Anti-LC3 (ab48394) and TOMM20 (ab56783) were acquired from Abcam, and anti-MAP2 (13-1500) was acquired from Invitrogen.

### Transient Middle Cerebral Artery Occlusion (MCAO)

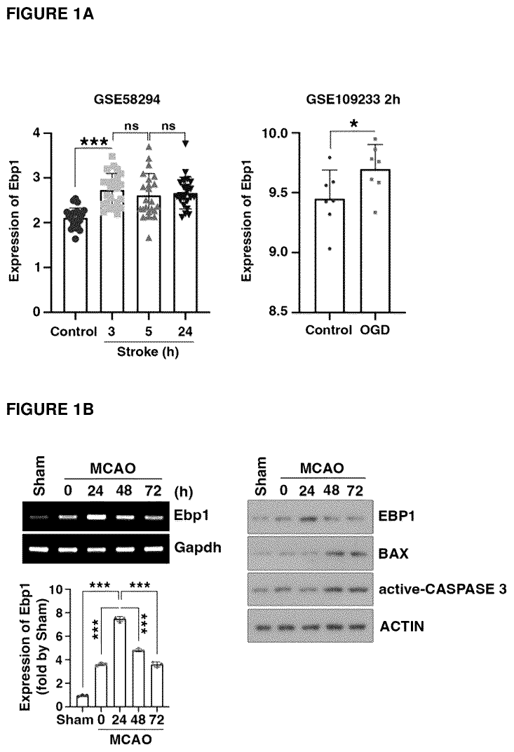

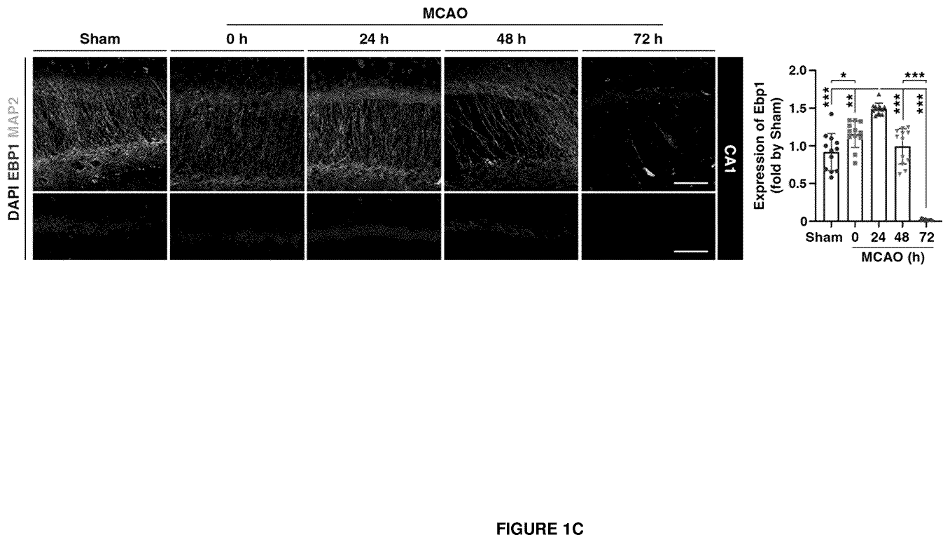

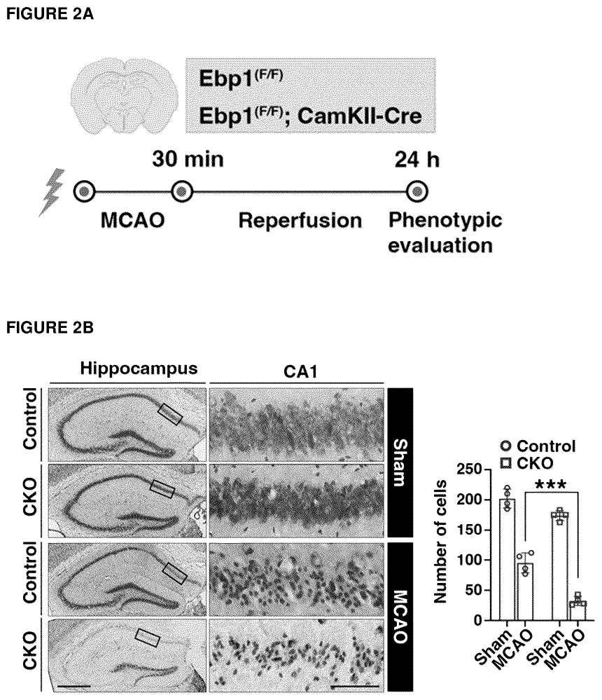

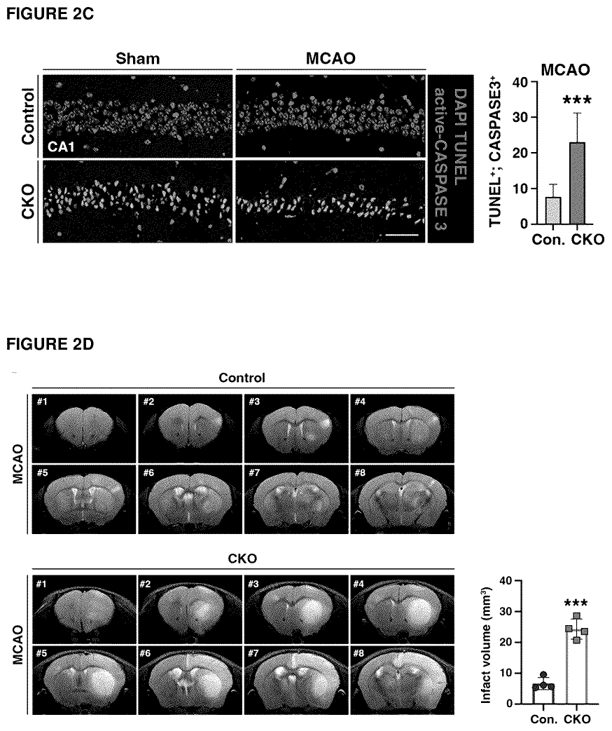

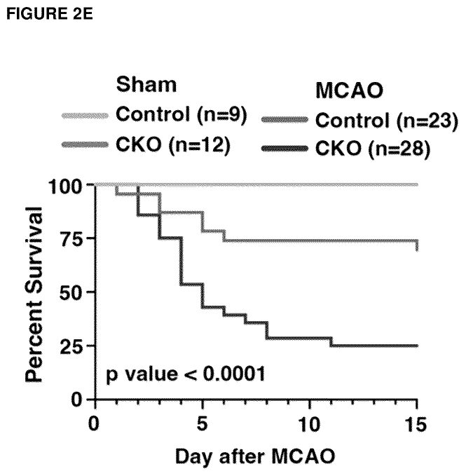

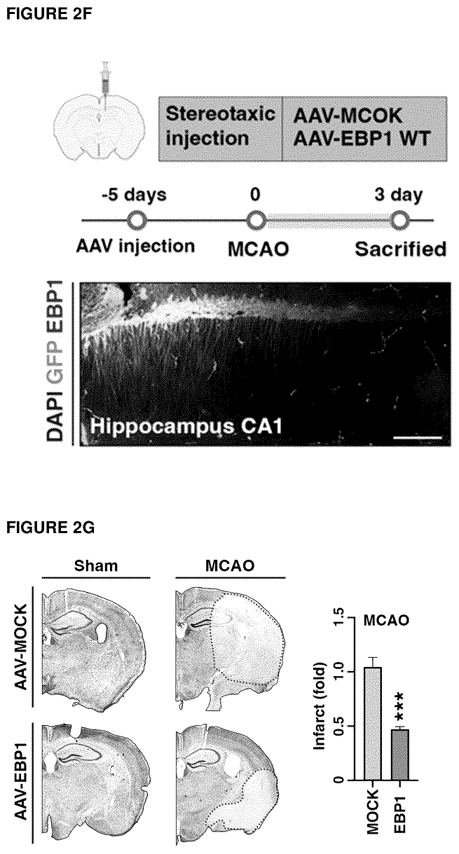

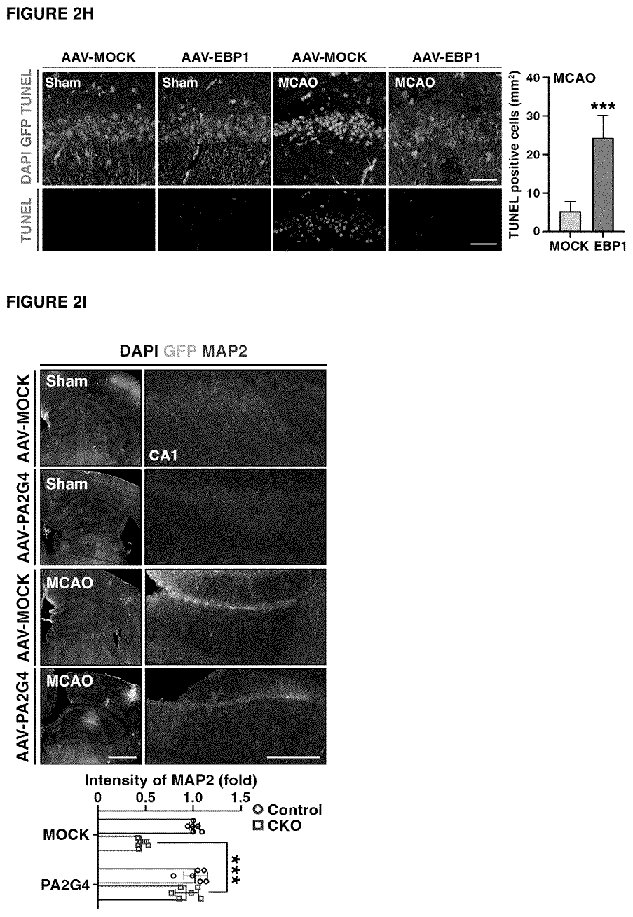

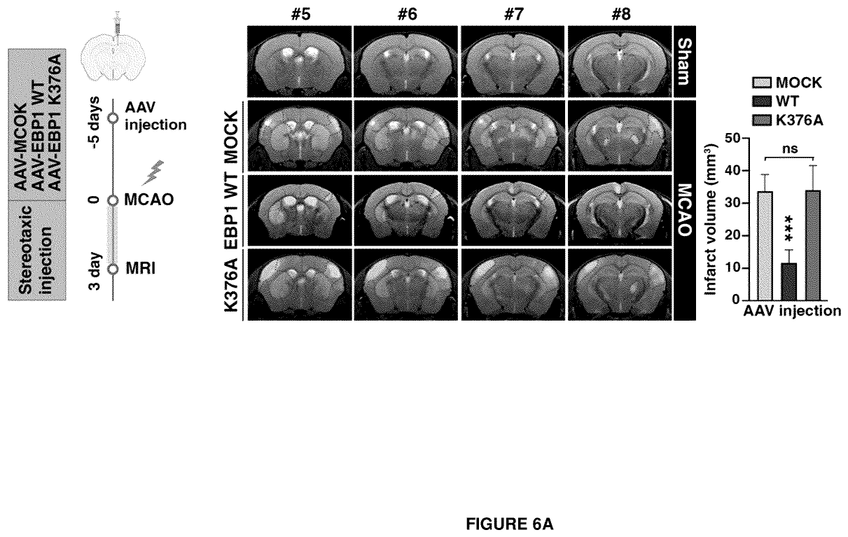

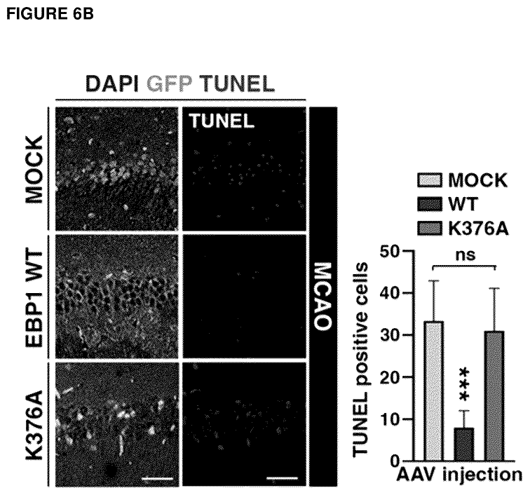

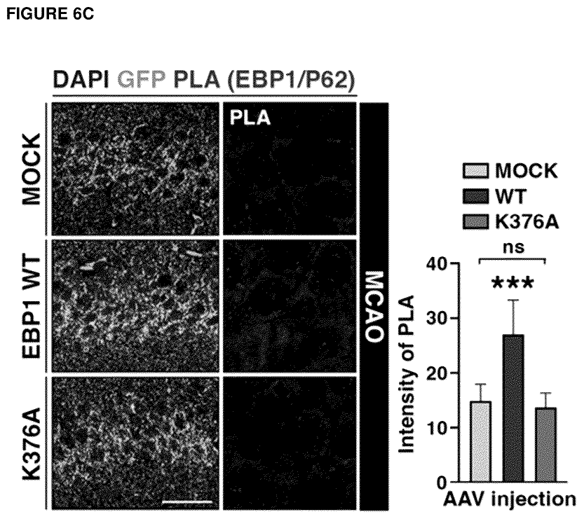

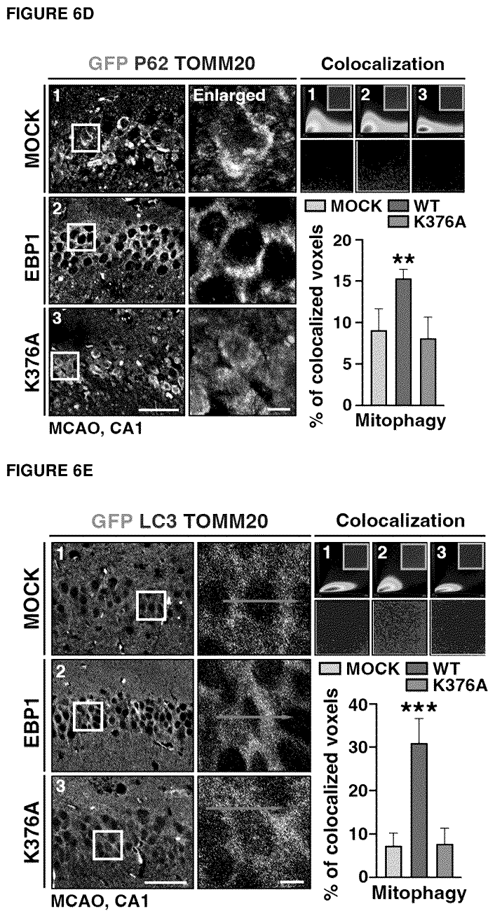

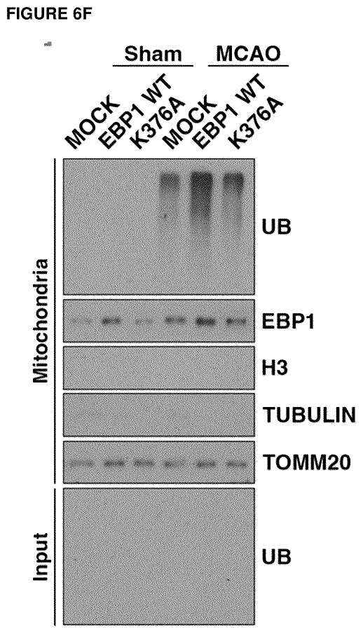

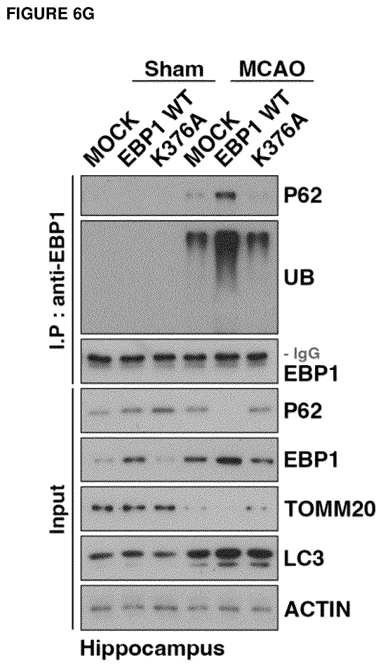

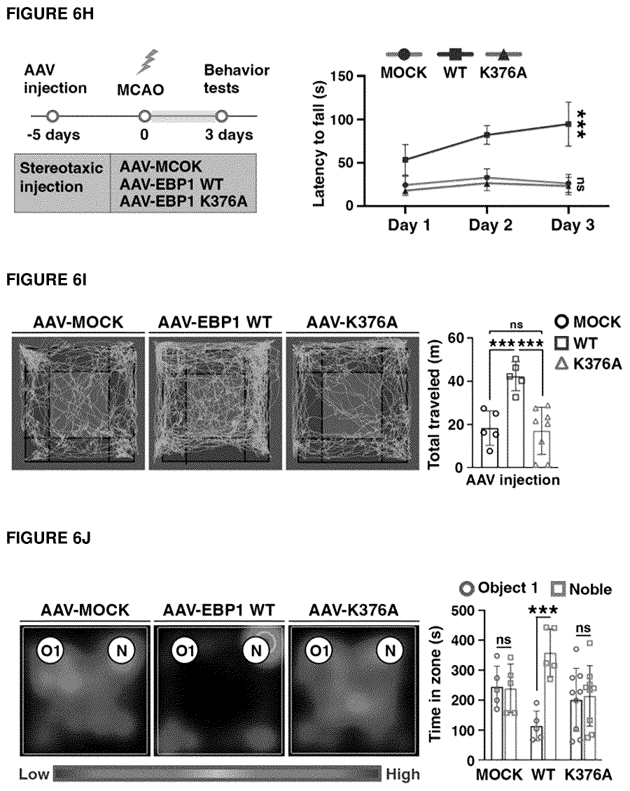

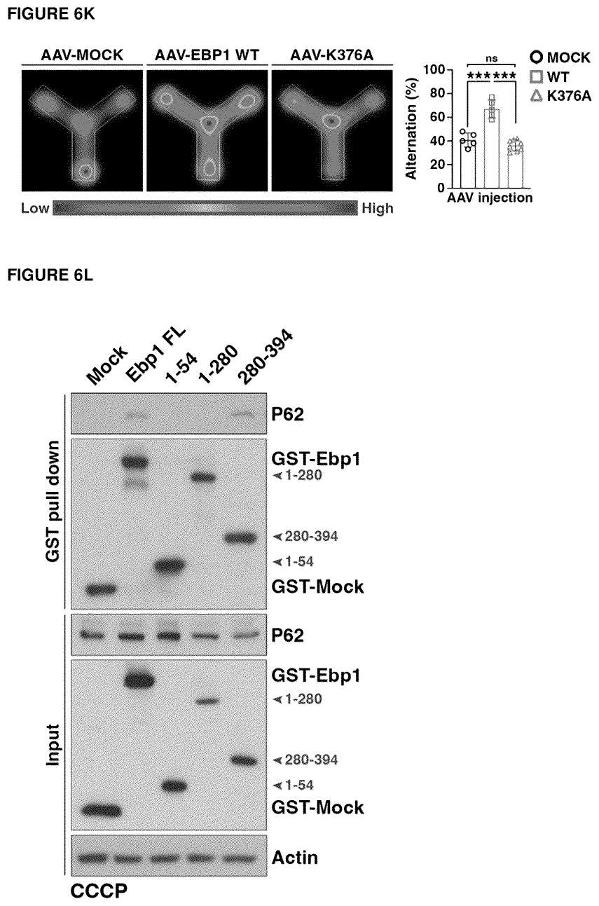

Mice (6-8 weeks old) were deeply anesthetized with isoflurane. The rectal temperature was maintained at 37° C. by using a feedback-controlled heating system. A midline ventral neck incision was made to expose the common carotid arteries for clamping to induce 30 (FIGS. 2 to 5) or 45 min (FIGS. 1 and 6) of ischemia. Then, the clamps were released for reperfusion. Sham control mice underwent the same operation without clamping.

### TUNEL Staining

TUNEL staining was performed on mouse hippocampi after ischemic stroke to detect apoptosis by using the In Situ Cell Death Detection Kit (Roche, catalog #11684795910). In addition, brain slices were treated according to the manufacturer's protocol.

### Ebp1 Cloning

To clone Ebp1 cDNA, total RNA was purified from HEK-293T cells, and cDNA was synthesized by using the PrimeScript 1st strand cDNA synthesis kit (Takara, 6110A). PCR was performed using a pair of nucleotide primers (Ebp1-F: 5′-gaattcatgtcgggcgagga-cgag-3′(SEQ ID NO: 5); and Ebp1-R: 5′-ctcgagtcagtccccagcttcattttct-3′(SEQ ID NO: 6)) corresponding to an open reading frame (ORF) based on NCBI database cDNA [Pa2g4, GenBank accession numbers NM_006191] to obtain full-length open reading frame (ORF) of Ebp1.

### Ebp1 Adeno-Associated Virus (AAV) Production

To express Ebp1 in AAV, Ebp1 full-length fragments obtained from HEK-293T cells were subcloned into pAAV-CMV vectors, and checked cloning by sequencing. AAV production was requested to be performed by the Virus center at Korea Institute of Science and Technology (KIST).

### Viral Delivery

Mice (6-8 weeks old) were anesthetized with 5% isoflurane, which was maintained at 2% throughout the operation. The top of the head was shaved, cleaned with 70% ethanol, and positioned into the stereotactic frame. A midline scalp incision was made, and a small craniotomy was performed using a drill mounted on the frame. The mice were injected with AAV2-MOCK (virus titer: 8.63×10¹² GC/mL), AAV2-EBP1 WT (virus titer: 2.41×10¹² GC/mL), or AAV2-K376A (virus titer: 2.77×10¹² GC/mL) into the left CA1 region of the hippocampus by using a 10-μL Hamilton syringe. AP: −1.8 mm; ML: −2.0 mm. DV: −1.9 mm.

### Immunoprecipitation and Immunoblotting

For GST pull-down assay, cells were rinsed with phosphate-buffered saline (PBS) and lysed in a buffer (50 mM Tris-CI, pH 7.4, 150 mM NaCl, 1 mM EDTA, 0.5% Triton X-100, 1.5 mM Na₃VO₄, 50 mM sodium fluoride), 10 mM sodium pyrophosphate, 10 mM beta-glycerophosphate, 1 mM phenylmethylsulfonyl fluoride (PMSF), and protease cocktail (Calbiochem, San Diego, CA). Cell lysates (0.5 to 1 mg of proteins) were incubated with Glutathione-Sepharose beads for 3 h at 4C. Then, the beads were washed in a lysis buffer, mixed with 2×SDS sample buffer, boiled, and analyzed by immunoblotting. Proteins were denatured, resolved by SDS-PAGE, and transferred onto nitrocellulose membranes (Pall Life Science, PortWashington, NY, USA). The membranes were blocked in 5% skim milk and incubated sequentially with primary antibodies and horseradish peroxidase (HRP)-conjugated secondary antibodies.

### Immunostaining and Nissl Staining

For IHC, mice were anesthetized, and perfused transcardially with saline, followed by 4% paraformaldehyde (PFA) injection. The brains were post-fixed in 4% PFA and incubated with 30% sucrose. Slices were cut and permeabilized using 0.25% Triton in PBS for 2 h, washed, and then blocked in PBS containing 2% bovine serum albumin for 1 h. The cells were immunostained by using primary antibodies overnight, and then incubated with secondary antibodies (Alexa Fluor-488 for green signal or Alexa Fluor-546 for red signal) at room temperature for 1 h. The nuclei were counter-stained with DAPI. The stained tissues were mounted using a mounting solution (Vector Laboratories, Burlingame, CA, USA). Z-stacked images were acquired using the Zeiss LSM 710 confocal microscope. All images were analyzed with identical parameters by using ZEN and ImageJ software.

For Nissl staining, paraffin-embedded sections were immersed in xylene and rehydrated through a series of decreasing concentrations of ethanol (50%, 70%, 90%, 95%, and 100%, 3 min each). The sections were washed in PBS and washed in 0.25% Cresyl Violet acetate for 10 min (Sigma, C5042; dissolved in distilled water containing 10 drops of glacial acetic acid per 100 mL of solution). The sections were treated with distilled water and dried and dehydrated in a series of an alcohol (50%, 70%, 90%, 95%, and 100% ethanol, 3 min each) and xylene (twice, 3 min each), and then mounted using Permount (Fisher Chemical, SP15-100) and left at room temperature overnight. Images were obtained using a slide scanner and analyzed by Aperio Imagescope (Leica).

Quantitative RT-PCR (qRT-PCR)

To compare mRNA levels, qRT-PCR. Total RNA was isolated from mice using the Takara mini BEST Universal RNA extraction kit (Takara, Japan). Then, cDNA was prepared from total RNA by reverse transcription by using oligo-dT primers (Takara, Japan). On the mice, qRT-PCR was performed using SsoFast EvaGreen Super Mix (Bio-Rad) according to the manufacturer's indications. A total reaction mixture of 20 μL was amplified in a 96-well PCR plate.

### MRI

MRI was performed on a horizontal bore 9.4 T/30-cm Burker BioSpec MR system (Billerica, MA, USA) at the Neuroscience Imaging Research (IBS) laboratory at Sungkyunkwan university. Anesthetized animals (1.5% isoflurane in air) were placed in a heated cradle where the temperature was maintained at 37° C. T2-weighted spin-echo images (TR/TE 4 1 5000/50 ms, slice thickness 0.5 mm, 15 slices) across the entire mouse brain were obtained. Each test group was blinded to genotype, and the infarct volume was calculated and measured using Horos (Horosproject.org).

### Hippocampal Slice Culture

MCAO modeling mice were euthanized. Brain slices of 200 μm thickness were obtained by vibratome sectioning (Leica VT1200, Leica Biosystems) in chilled MEMp (50%, vol/vol, minimum essential medium, 25 mM HEPES, and 2 mM glutamine without antibiotics), adjusted to pH 7.2-7.3 (with 1 M NaOH). The slices were transferred onto semi-porous membrane inserts (Millipore) from MEMp. The slices were fixed with 4% PFA in PBS, and the fixed slices were washed and collected from the inserted membrane. The mitophagy was determined using a mitophagy detection kit (DOJINDO, Japan, MD01).

### Behavioral Testing

All behavioral tests were performed using age- and gender-matched mice.
Novel object recognition test: The novel object recognition test was performed in standard mouse cages. This test consisted of three phases: habituation, familiarization, and test phases. In the habituation phase, mice were acclimated in an empty mouse cage to adapt to the environment for 10 min. The next day, in the familiarization phase, mice were placed in the same cage with two identical objects, and the time during which the mouse nose touched the object or was oriented toward the object and came within 2 cm of the object was measured for 10 min. In the first test phase, 24 h after the familiarization phase, one of the two objects was replaced with a new object, and the same measurements were recorded for 10 min. The movement of each mouse was recorded and analyzed using the Video tracking software EthoVision XT14 (Noldus, The Netherland).
Y-maze: The Y-maze activity was tested using a symmetrical Y-maze. Each mouse was placed in the side wall of an arm of the maze and allowed to explore the maze for 10 min. The movement of each mouse was recorded and analyzed using the Video tracking software EthoVision XT14 (Noldus, The Netherland).
Open field: The open field test was used to assess anxiety and exploratory behaviors. Each mouse was placed near the wall of the open field area (44.5×44.5 cm). The area was separated into two zones: the center (28.5×28.5 cm) and the periphery (surrounding the center). The open field test was performed for 20 min. The movements of the mice were recorded and analyzed using the animal activity meter Opto-Varimex-5 Auto-Track (Columbus, OH, USA).
Rotarod test: An accelerating rotarod (Ugo Basile, Italy) was used to assess motor coordination and balance. Mice were placed on the cylinder, which slowly accelerated from 4 to 40 rpm over a 5-min test session. The task required the mice to walk to remain on top of the rotating rod.

### Data Analysis and Statistical Analysis

Numerical data are presented as means±S.E.M. The significance of data for comparison was obtained by Student's two-tailed unpaired t test, One-Way ANVA, and Two-Way ANOVA, and significance levels were displayed as * (p<0.05), ** (p<0.01), ***(p<0.001).

### EXPERIMENTAL RESULTS

### Experimental Example 1: Up-Regulation of EBP1 and Alleviation of Neuron Loss after Cerebral Ischemic Injury

Since the loss of Ebp1 causes massive neuron loss in the brain and EBP1 plays an important role in neuronal survival, the present inventors hypothesized that EBP1 may contribute to neuroprotection after cerebral IR injury.

Up-regulated expression of Ebp1 was recorded in the publicly accessible database of stroke patients (GSE58294), oxygen-glucose deprivation (OGD, GSE109233), and ex vivo models of cerebral ischemia (FIG. 1A).

To investigate the potent role of EBP1 in ischemic damage, the present inventors performed transient middle cerebral artery occlusion (MCAO) in mice. As a result, mRNA and protein levels of EBP1 were highly upregulated within 24 h and then gradually diminished for 72 h. The EBP1 levels were inversely correlated with those of active caspase-3 or Bax, which are hallmarks of apoptosis (FIG. 1B).

After stroke was induced in wild-type mice, it was investigated whether the Ebp1 RNA and protein expression was increased in the hippocampus. As a result of immunohistochemistry (IHC), in the mouse brain, the EBP1 expression was transiently increased in the CA1 region of the hippocampus within 24 h after MCAO and decrease over time with significant neuronal loss (FIG. 1C). The results suggest that the upregulation of EBP1 during the transient period prevents neuronal death, but with prolonged MCAO, the EBP1 expression decreases and no longer protects neurons.

### Experimental Example 2: Exacerbation of Brain Damage by Ischemia reperfusion (IR) in Ebp1 deficiency

To determine the physiological roles of EBP1 under ischemic damage, forebrain-specific Ebp1-conditional knockout mice (CamKII-Cre; Ebp1^{flox/flox}; hereinafter, Ebp1-CKO) were generated by crossing Ebp10^{flox/flox} (hereinafter, control) with CaMKIla-Cre driver. It was verified through mRNA and IHC analyses that the EBP1 expression was successfully removed in the hippocampus and cortex but not in the cerebellum (FIG. 1C).

These Ebp1-CKO and control mice were used to perform conduct cerebral IR injury (FIG. 2A). The Ebp1 deficiency significantly exacerbated ischemic brain injury as revealed by a severe neuron loss in the CA1 region compared with MCAO-operated control mice (FIG. 2B).

In addition, the neuronal death marked by active caspase-3 and TUNEL staining was significantly increased in CA1 of CKO mice (FIG. 2C). T2-weighted magnetic resonance imaging (MRI) allowed to visualize the damaged brain structure and quantify the brain infarct volume by IR, revealing that Ebp1-CKO mouse brains were more vulnerable ischemic injury than control mouse brains (FIG. 2D). Hence, the Ebp1 deficiency reduced the survival of mice compared with the control mice after MCAO (FIG. 2E) (p<0.0001).

To validate that an increase in the ischemic brain injury found in Ebp1-CKO mice is actually due to Ebp1 deficiency and Ebp1 is responsible for neuroprotection, the present inventors generated adeno-associated virus serotype 2 (AAV2) expressing GFP-EBP1 or GFP-mock.

The in vivo injection was conducted into the prospective IR injury site in the CKO and control mice 5 days before MCAO (FIG. 2F).

The timing of viral injection enables AAV2 to express from day 1 after IR injury. The success of the adenoviral delivery into the in vivo injection was verified using time series IHC analysis of brain sections (FIG. 2G).

The expression of GFP-EBP1 explicitly reduced the brain infarct volume after IR (FIG. 2H).

Additionally, the frequency of MAP2-positive neurons prominently increased in the brain of CKO mice expressing AAV2-GFP-EBP1, whereas neurons in the brain of MCAO-operated CKO mice expressing AAV2-GFP-mock still showed a largely reduced intensity of MAP2 (FIG. 2I). Accordingly, the reintroduction of EBP1 into CKO mice restored the movement of the mice after MCAO, whereas CKO mice expressing GFP-control rarely showed motion.

Moreover, TUNEL-positive cells were markedly reduced in MCAO-operated CKO brains expressing AAV2-GFP-EBP1 (FIG. 2H). In contrast, AAV2-GFP-mock expression could not prevent neuronal death in IR-injured CKO mouse brains (FIG. 2I). Therefore, the phenotypic rescue and neuroprotection by the reinstatement of EBP1 implies that the depletion of Ebp1 is the major cause of Ebp1-CKO mice vulnerability during IR injury, suggesting the potential for a therapeutic role of EBP1 function in the in ischemic brains.

### Experimental Example 3: Need for EBP1 in Ischemia Reperfusion Induced Mitophagy

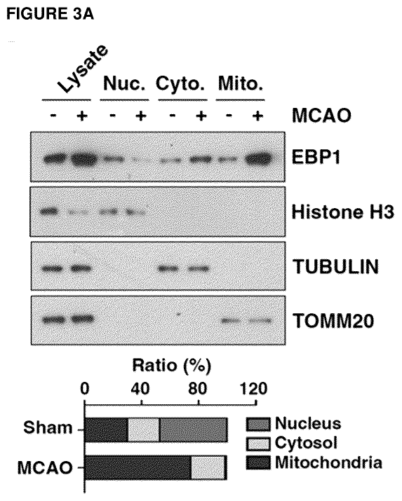

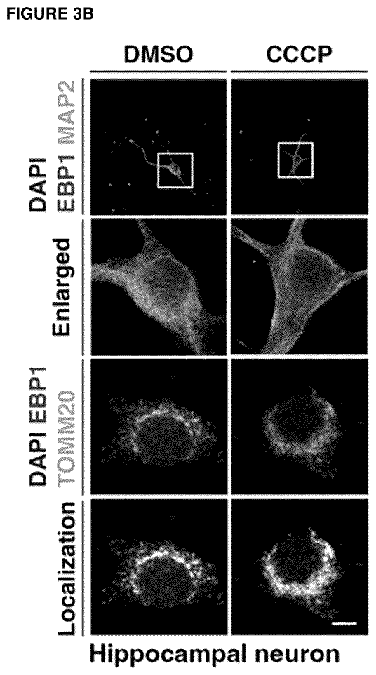

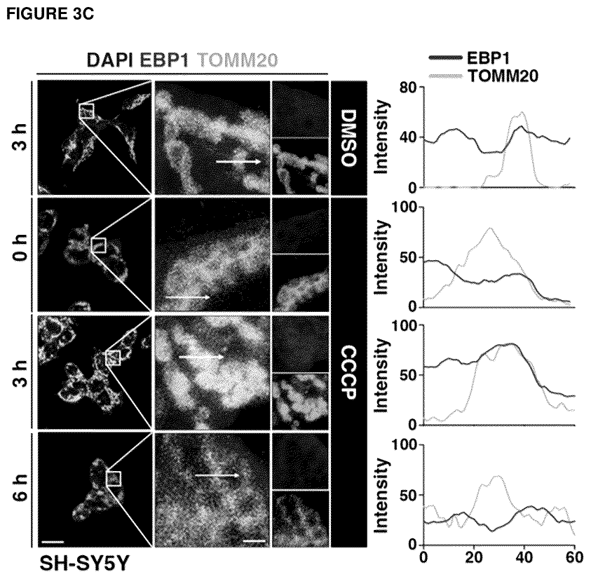

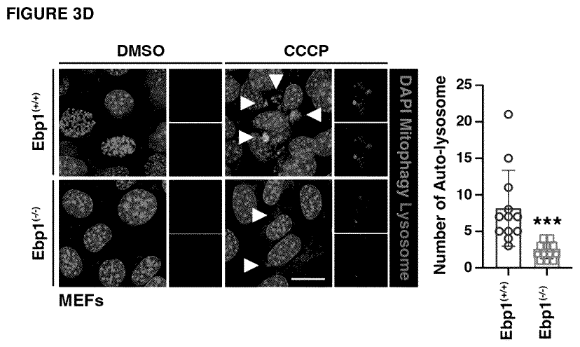

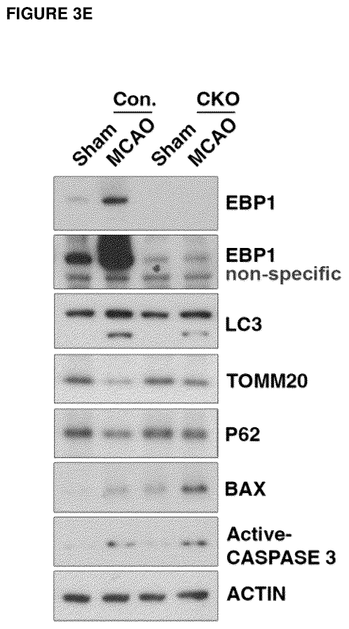

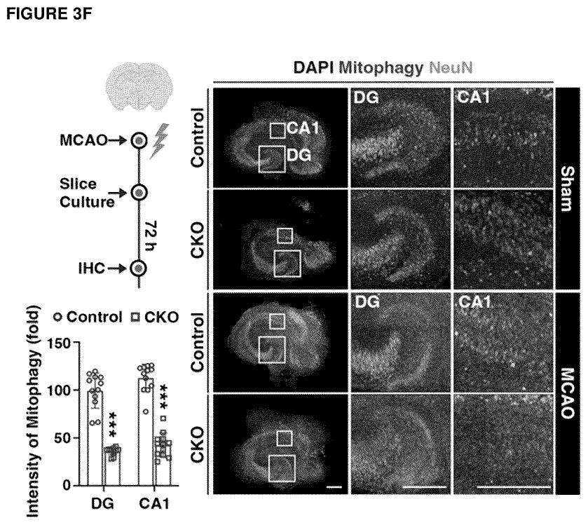

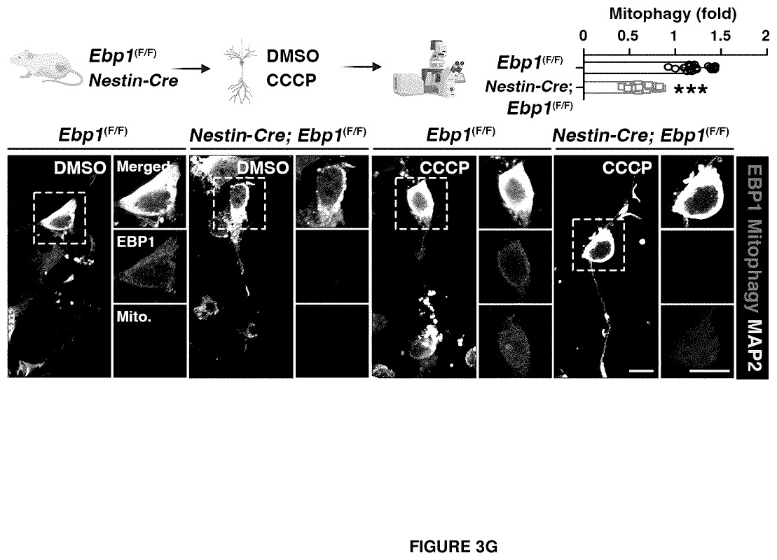

During ischemia reperfusion (IR), mitophagy inhibition exacerbates brain damage, whereas stimulation of mitophagy is known to be beneficial to neuronal survival in the rapid period of reperfusion. The expression of EBP1 was upregulated at early time points after MCAO to alleviate the neuron loss (FIGS. 1 and 2), and EBP1 was notably translocated from the cytoplasm to the mitochondria in the mouse brain after MCAO while the nuclear localization was reduced (FIG. 3A). Therefore, the present inventors hypothesized that EBP1 can contribute to IR-induced mitophagy.

To test this hypothesis, mitophagy was induced by treating primary cultured hippocampal neurons or SH-SY5Y neuronal cells with carbonyl cyanide m-chlorophenyl hydrazine (CCCP), a mitochondria uncoupler.

As a result, EBP1 was translocated to depolarized mitochondria as shown by the co-localization with TOMM20, a mitochondria marker protein, after 3 h treatment with CCCP. This translocation declined after 6 h of treatment (FIGS. 3B and 3C), and EBP1 was transiently accumulated in the damaged mitochondria.

Next, as a result of treating Ebp1^{(+/+)} and Ebp1^{(-/-)} MEF with CCCP to determine the physiological role of EBP1 mitochondria translocation, approximately 50% less mitophagy induction, determined by the number of autolysosomes, was found in Ebp1^{(-/-)}. These results indicate that the Ebp1 deficiency injured mitophagy induction compared with Ebp1^{(+/+)} MEF (FIG. 3D).

To further investigate the involvement of EBP1 in mitophagy induction, the present inventors performed MCAO in the Ebp1-CKO and control mice. There was a marked decrease in the immunoreactivity of TOMM20 combined with an increase in LC3 demonstrating mitophagy induction in hippocampal CA1 of control mice. However, the present inventors verified that the LC3 intensity was relatively low and the TOMM20 intensity was partially reduced in the hippocampus of Ebp1-CKO mice, indicating that the mitophagy induction was impaired (FIG. 3E).

It was also verified through immunoblotting that, compared with control mice, MCAO-treated CKO brains exhibited reduced mitophagy induction with decreased LC3 intensities and relatively lower TOMM20 intensities, but exhibited increased cell death with high levels of Bax and active caspase-3 (FIG. 3E). These data imply that transient mitochondria accumulation of EBP1 upon IR injury can be involved in mitophagy induction for neuronal protection.

To clarify whether EBP1 participated in IR-induced mitophagy, the present inventors were conducted ex vivo culture of hippocampal slices from ischemic-damaged brains. Actually, in the absence of EBP1, the mitophagy induction was alleviated in the hippocampus including DG and CA1 regions, compared with the hippocampus of control mice, but the sham-treated group did not induce mitophagy in the hippocampus of control or CKO mice (FIG. 3F). Mitophagy was induced using CCCP in primary hippocampal neurons lacking EBP1, but mitophagy was confirmed to be reduced compared to the control group (FIG. 3G). Accordingly, the data suggest that the neuroprotective effect of EBP1 may be caused by mitophagy induction in IR injury.

### Experimental Example 4: Role of EBP1 as PARKIN Substrate in Mitophagy Induction

The present inventors investigated how EBP1 functions for mitophagy induction in IR injury. EBP1 is known to be implicated in the ubiquitin-proteasome system by either being a substrate of E3 ligase or connecting E3 ligase to the substrate. After mitochondria depolarization, PARKIN is a key E3 ligase that, in coordination with PINK1, triggers mitophagy to eliminate damaged mitochondria, and this induces substrate ubiquitination in OMM and utilizes ubiquitin chains as a molecular signal to recruit mitophagy machinery. As a recent proteomic analysis validated that EBP1 is a putative interacting partner of PARKIN, it was speculated whether EBP1 is involved in PARKIN-dependent mitophagy induction.

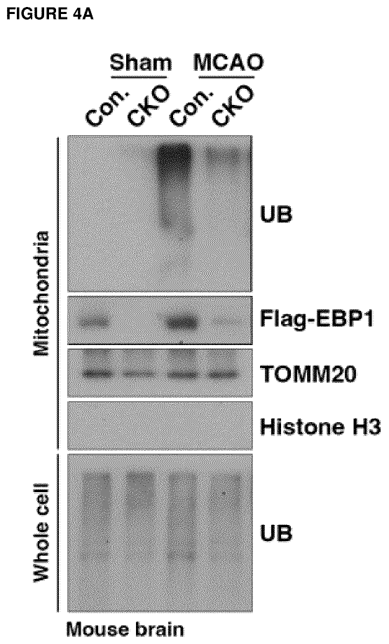

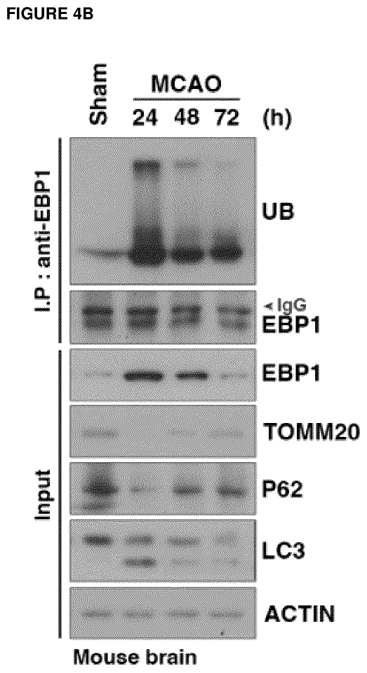

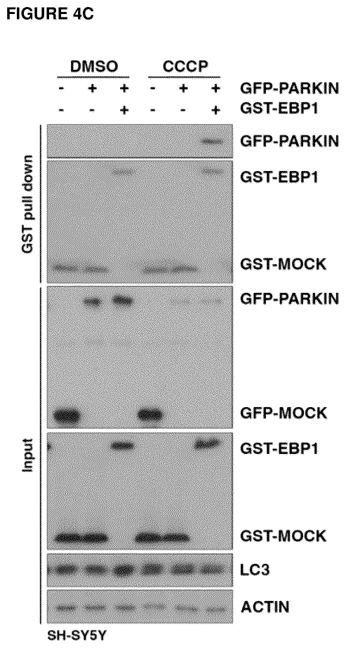

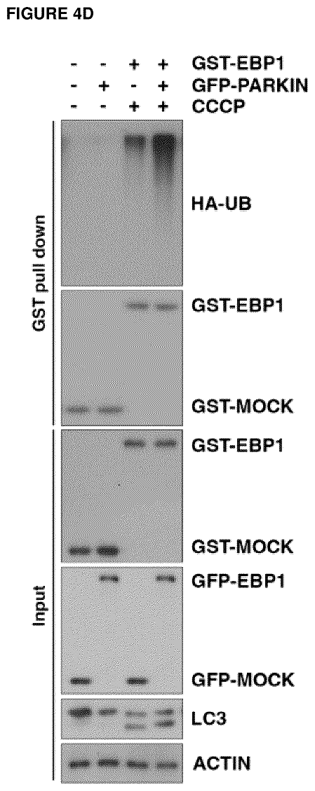

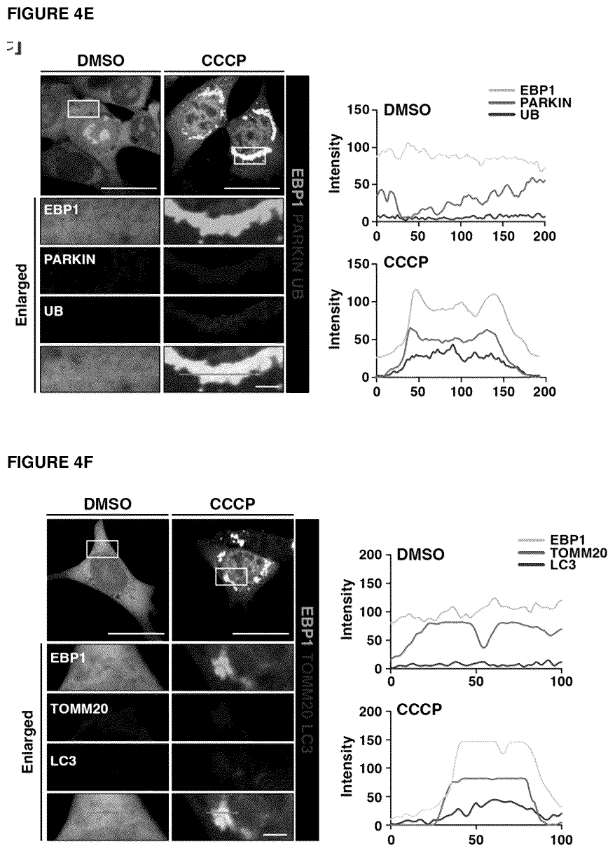

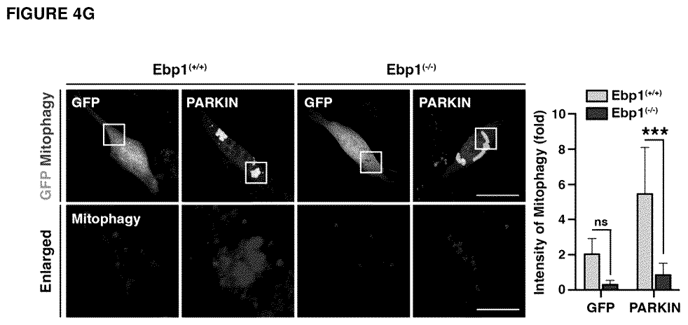

Under MCAO operation of CKO and control mice, the present inventors found that the ubiquitination of mitochondrial fractions in the brains of controls was marked, whereas this ubiquitination was dramatically reduced in the CKO mouse hippocampus (FIG. 4A). In addition, EBP1 itself was ubiquitinated upon IR injury, and EBP1 ubiquitination was particularly enhanced at 24 h when the levels of proteins with protective ability peaked with mitophagy induction, which was shown to be increased LC3, and reduced P62 and TOMM20 intensities (FIG. 4B). This suggests that the effects of EBP1 are related to PARKIN-dependent mitochondria degradation.

Therefore, EBP1 directly bound to PARKIN under the mitochondria damage induced by CCCP treatment in SH-SY5Y cells, whereas no interaction between PARKIN and EBP1 was observed in the control vehicle treatment (FIG. 4C).

The ubiquitination assay after co-transfection of GST-EBP1 with GFP-PARKIN in HEK293 cells showed that EBP1 was ubiquitinated upon mitophagy induction by CCCP treatment and this ubiquitination increased by over-expression of PARKIN. However, the levels of ubiquitinated EBP1 were not altered (FIG. 4D).

Upon mitophagy induction by CCCP treatment in hippocampal neuronal HT-22 cells, EBP1 was accumulated with PARKIN in the mitochondria, revealing evident ubiquitination as well as mitophagy induction shown by increased LC3 and reduced TOMM20 intensities (FIGS. 4E and 4F).

Despite the over-expression of GFP-PARKIN, Ebp1^{(-/-)} MEF cells did not exhibit mitophagy. However, Ebp1^{(+/+)} MEF cells expressing GFP-PARKIN clearly induced mitophagy (FIG. 4G). Accordingly, the above results verified that EBP1 is a substrate for PARKIN E3 ligase and contributes to PARKIN-mediated mitophagy induction.

### Experimental Example 5: Mechanism of Mitophagy Induction of EBP1 PARKIN-Mediated Ubiquitination of Lysine (K) 376 in EBP1 Contributes to Recruitment of Mitophagy Adaptor Protein P62

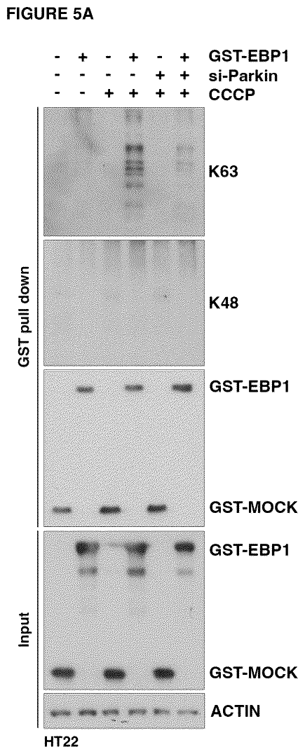

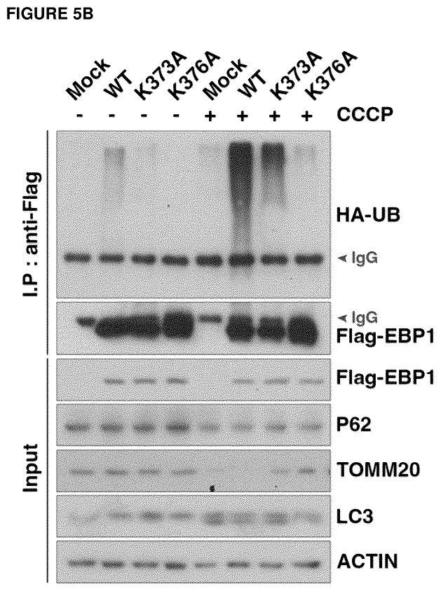

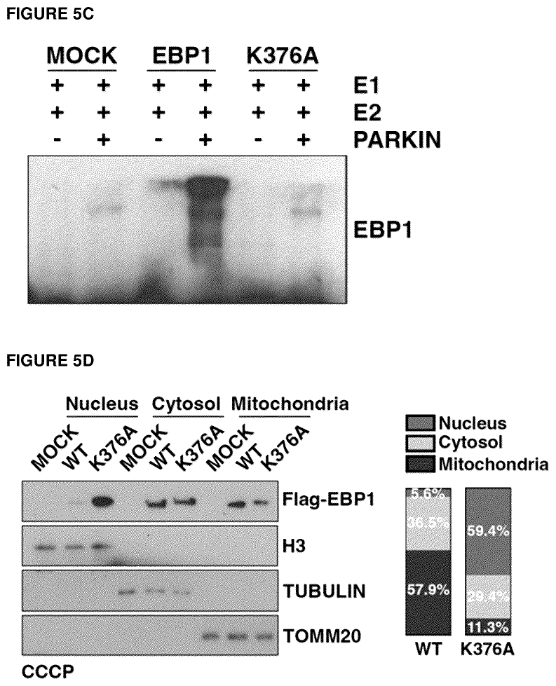

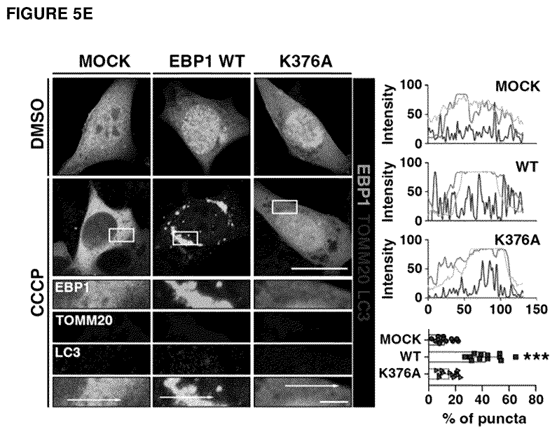

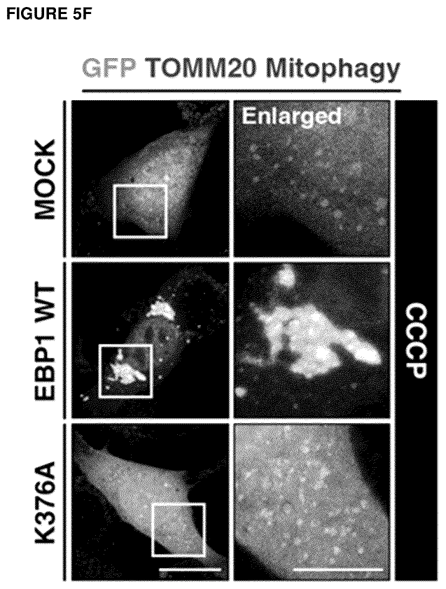

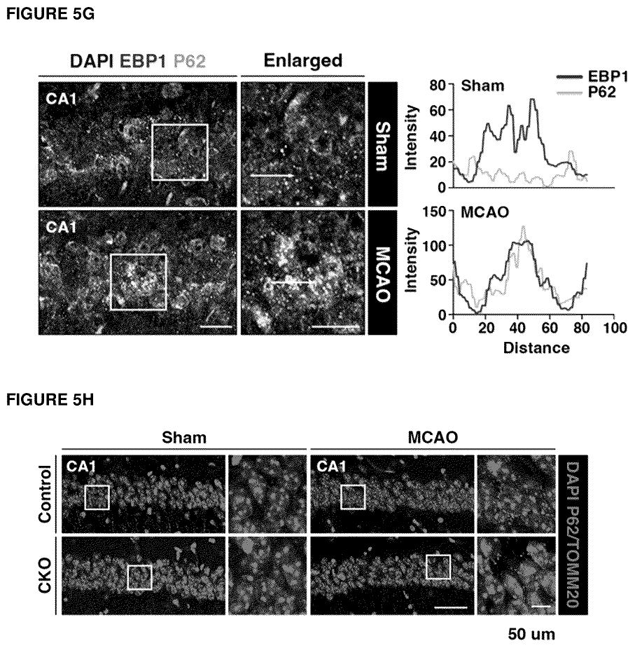

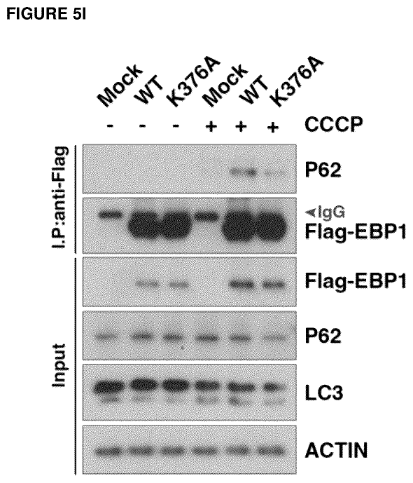

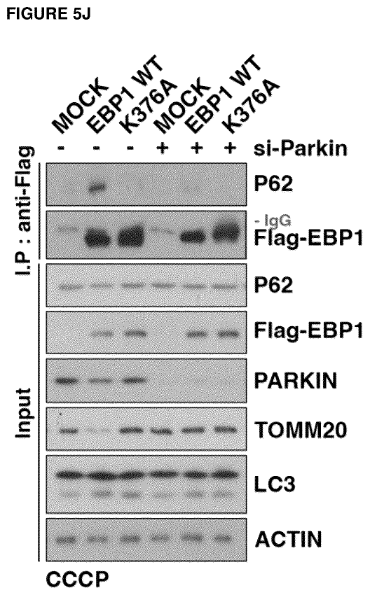

The present inventors predicted K373 and K376 as putative ubiquitination sites by using the Phosphosite Plus database. Thereafter, the present inventors generated a EBP1 mutant by substituting K373 or K376 with alanine (A). Under mitochondria depolarization conditions by CCCP treatment for 1 h, EBP1 WT and 373A mutant, but not K376A mutant, were notably ubiquitinated compared with the control vehicle-treated group, implied that EBP1 was translocated to the mitochondria and ubiquitinated at K376 in the damaged mitochondria (FIG. 5A).

The in vitro ubiquitination assay performed using purified E1, E2, and PARKIN along with purified EBP1 WT and K376A clearly showed that K376 is the residue corresponding to PARKIN-mediated ubiquitination (FIG. 5B).

The present inventors also verified through EBP1 sequencing that K376 is conserved throughout evolution in many species. Additionally, three-dimensional modeling using the AlphaFold program (https://alphafold.ebi.ac.Uk) verified that K376 is located at the exposed helical domain of the protein and K376 can be a strong target for PARKIN E3 ligase.

In contrast EBP1-WT, K376A mutant, which was not ubiquitinated by PARKIN, did not contribute to mitophagy induction along with delocalization at the mitochondria (FIG. 5D), suggesting that K376 ubiquitination in EBP1 by PARKIN is crucial for mitophagy induction.

PARKIN predominantly forms K48- and K63-linked chains during mitophagy to eliminate mitochondrial substrates from the OMM to recruit autophagy receptors, such as P62, for autophagosome formation. Therefore, the present inventors examined the type of ubiquitin chains in EBP1. The in vitro ubiquitination assay validated the K63-linked ubiquitination in EBP1, but there was no EBP1 K48-linked ubiquitination, reflecting no changes in EBP1 protein levels upon CCCP-induced mitophagy (FIG. 5C, 4th lane). Moreover, siRNA silencing of Parkin abolished the K63-linked ubiquitination of EBP1, confirming that PARKIN induced K63 ubiquitination of EBP1 in HT-22 cells (FIG. 5C, 6th lane).

After it has been established that K63 chain ubiquitination of the K376 residue of EBP1 induced mitophagy, the present inventors considered whether PARKIN mediated EBP1 ubiquitination to recruit autophage receptor proteins. The siRNA Silencing of several mitophagosome receptor proteins, such as p62, Optn, and Ndp52, while having or not Flag-EBP1, showed that forcible expression of EBP1 can induce mitophagy in the absence of Optnor Ndp52. However, when p62 was depleted, the over-expression of EBP1 contributed to only mitophagy induction.

Consistent with these observation results, the protein ligation assay (PLA) validated that the association of EBP1 with P62 was significantly enhanced in the CA1 region of the mouse brains treated with MCAO (FIG. 5E).

MCAO resulted in significant mitochondrial accumulation of P62 in the CA1 of the hippocampus, and 80% or more of EBP1-expressing cells were co-localized with P62. These results suggest that EBP1 contributed to the recruitment of P62 in the damaged mitochondria (FIG. 5F).

Additionally, in MCAO-operated CKO mice, the binding affinity between P62 and TOMM20 was decreased compared with MCAO-treated control mice (FIG. 5G).

Furthermore, in SH-SY5Y cells, EBP1 WT physically interacted with P62 under CCCP treatment, but the K376A mutant did not. EBP1 WT or K376A mutant did not interact with P62 without CCCP treatment (FIG. 5I).

However, when the expression of PARKIN was silenced by siRNA, EBP1-WT or K376A did not interact with P62, regardless of CCCP treatment (FIG. 5J).

Altogether, these data suggest that under ischemic injury, EBP1 ubiquitinated at K376 by PARKIN serves as alinker for recruiting the adaptor protein P62 in the CA1 region of the hippocampus and contributed to mitophagy induction.

### Experimental Example 6: Essentiality of EBP1 K376 Ubiquitination in Mitophagy Induction and Neuroprotection Upon IR Injury

To determine the in vivo effects of EBP1 ubiquitination upon IR injury, AAV2-GFP-EBP1-K376A or EBP1-WT was injected into the prospective IR injury site in the mouse brain 5 days before the MCAO operation (FIG. 6A).

The brain infarct volume of IR injury shown by MRI was markedly decreased by the over-expression of EBP1-WT, but not by that of K376A mutant, compared with the infarct volume in the control MOCK (FIG. 6A)

TUNEL analyses showed that neuronal death was highly increased in the AAV2-GFP-EBP1-K376A-expressing hippocampi (up to 60%) compared with AAV2-GFP-EBP1-WT-expressing hippocampi (up to 10%) (FIG. 6B). These results suggest that the K376A mutant impairs the neuroprotective effect of EBP1 in vivo against MCAO.

The immunoreactivity intensities of TOMM20 and P62 were diminished but the immune response intensity of LC3 was increased in the brain of EBP1-WT-expressing mice after MCAO, whereas the immunoreactivity intensities for these markers of mitophagy induction were reversed in the brain of K376A-expressing mice (FIGS. 6D and 6E).

Moreover, in situ PLA signals were elevated in MCAO mouse brains injected with EBP1-WT to visualize the association of P62 and EBP1, whereas PLA positive signals were not changed after MCAO in the EBP1-K376A-expressing mouse brains (FIG. 6C). These results indicate that K376 ubiquitination of EBP1 is required for P62 recruitment and mitophagy induction.

Importantly, EBP1-WT-expressing mouse brains following MCAO revealed apparent ubiquitination of mitochondria and EBP1 ubiquitination coupling with P62. In contrast, K376A-expressing mouse brains after MCAO showed reduced ubiquitination of mitochondria but not P62 binding or EBP1 ubiquitination (FIGS. 6F and 6G). Therefore, EBP1 K376 ubiquitination may be an essential step for mitophagy induction in the early stages of MCAO.

To further investigate the functional relevance of EBP1 K376 ubiquitination and mitophagy induction, a series of behavioral tests were performed (FIG. 6H).

The rotarod and open-field tests showed that AAV2-GFP-EBP1-K376A expressing mice did not rescue locomotor defects, whereas the impaired ambulatory abilities were greatly recovered in AAV2-GFP-EBP1-WT-expressing mice compared with AAV2-GFP-mock-expressing mice (FIGS. 6H and 6I).

In the novel object recognition test, AAV2-GFP-EBP1-WT-expressing mice spent more time exploring the novel object (N1) than the older one (O1), whereas the times for exploring either N1 or O1 by the GFP-EBP1-K376A-expressing mice were similar to those by control expressing mice (FIG. 6J). Therefore, the recognition memory that was obviously impaired by MCAO was improved by the expression of EBP1-WT but not EBP1-K376A.

In the Y-maze test, EBP1-K376A-expressing mice with MCAO showed a reduced spontaneous change rate (39% reduction), which was similar to that of control mice with MCAO, whereas the EBP1-WT expression restored this dysfunctional working memory (FIG. 6K).

Collectively, these data showed that the over-expression of EBP1 before IR injury can ameliorate the functional impairments occurring by MCAO, whereas the interruption of PARKIN-directed ubiquitination of EBP1 cannot contribute to the restoration to IR-elicited behavior impairments.

Furthermore, the present inventors verified 280-394 amino acids, which correspond to a small unit capable of binding to P62 to induce mitophagy in the EBP1 protein. However, as shown in the above experimental examples, it can be predicted that a smaller unit of EBP1 containing K376 amino acid can also be used in the present invention, from the results that the K376A mutation of EBP1 inhibited EBP1 K376 ubiquitination.

What is claimed is:

1. A method for treating ischemic cerebrovascular disease, the method comprising administering to a subject ErbB3-binding protein 1 (EBP1) protein or a polynucleotide encoding EBP1 protein.

2. The method of claim 1, wherein the EBP1 protein includes the amino acid sequence represented by SEQ ID NO: 1.

3. The method of claim 2, wherein the EBP1 protein includes the amino acid sequence represented by SEQ ID NO: 2.

4. The method of claim 1, wherein the polynucleotide encoding EBP1 protein includes the nucleotide sequence represented by SEQ ID NO: 3.

5. The method of claim 1, wherein the polynucleotide encoding EBP1 protein includes the nucleotide sequence represented by SEQ ID NO: 4.

6. The method of claim 1, wherein the polynucleotide is naked DNA, or is contained in a gene carrier.

7. The method of claim 6, wherein the gene carrier is a vector.

8. The method of claim 6, wherein the vector is a plasmid vector, a cosmid vector, or a viral vector.

9. The method of claim 8, wherein the viral vector is selected from the group consisting of an adenoviral vector, a retroviral vector, a lentiviral vector, and an adeno-associated viral vector.

10. The method of claim 9, wherein an adeno-associated virus (AAV) for the adeno-associated viral vector is AAV1, AAV2, AAV5, or AAV6.

11. The method of claim 1, wherein the ischemic cerebrovascular disease is selected from the group consisting of transient ischemic attack, reversible ischemic neurological deficit, and obstructive stroke.

12. The method of claim 11, wherein the obstructive stroke is caused by blood clots or embolisms.

13. The method of claim 11, wherein the stroke is an acute stroke within 7 days of the onset.
